(19) United States
(12) Patent Application Publication
(10) Pub. No.: US 2024/0261532 A1
(43) Pub. Date: Aug. 8, 2024

(54) VIRTUAL REALITY-BASED PSYCHOTHERAPY SYSTEM WITH REAL-TIME ELECTROENCEPHALOGRAM FEEDBACK

(71) Applicant: UNIVERSITY OF KANSAS

(72) Inventors: Christopher TACCA; Barbara A. Kerr, Eudora, KS (US)

(73) Assignee: UNIVERSITY OF KANSAS, (US)

(21) Appl. No.: 18/566,926

(22) PCT Filed: Jun. 3, 2022
(86) PCT No.: PCT/US2022/032164

§ 371 Date: Dec. 4, 2023

Related U.S. Application Data
(60) Provisional application No. 63/197,191, filed on Jun. 4, 2021.

(51) Int. Cl. A61M 21/00; A61B 5/00; A61B 5/16; A61B 5/375; G06T 19/00

(52) U.S. Cl. CPC A61M 21/00; A61B 5/0006; A61B 5/0022; A61B 5/165; A61B 5/375; A61B 5/6803; A61B 5/744; A61B 5/7465; G06T 19/00; G06T 2219/024

(57) ABSTRACT

A remote psychotherapy system includes an environment. A patient selects a patient avatar. The patient provides input for a provider avatar, which may be influenced by one or more Jungian archetypes. The patient wears a virtual reality simulator having a head mounted device. The virtual reality simulator includes a sensor that collects biofeedback information. The biofeedback information is used to determine state information about the patient, such as an emotional state of the user. The state information and/or the biofeedback information is transmitted to the provider and presented to the provider in a graphical user interface (GUI) on a provider virtual reality device.

### CROSS-REFERENCE TO RELATED APPLICATIONS

This application claims priority to and the benefit of U.S. Provisional Patent Application No. 63/197,191, filed on Jun. 4, 2021, which is hereby incorporated by reference in its entirety.

### BACKGROUND

### Background and Relevant Art

Patients with depression and anxiety can lack access to immersive and personalized therapeutic care. Geographically and psychologically isolated patients can benefit from a remote therapy alternative that is non-stigmatizing and accessible.

### BRIEF SUMMARY

In some embodiments, electroencephalogram (EEG) information is collected via a head-mounted device (HMD) that also houses a near-eye display (NED) to provide visual information and/or stimulation to the patient during the psychotherapy session. A biofeedback on the HMD measures biofeedback information correlated to a communication between the provider avatar and the patient avatar or the virtual environment.

In some embodiments, a method for remote psychotherapy includes preparing a virtual environment for a remote therapy session. A provider avatar is provided for a provider and a patient avatar for a patient. The method includes receiving biofeedback information from said patient correlated to a communication to the patient from the provider avatar or the virtual environment and determining state information about said patient based on the biofeedback information. The state information is provided to said provider.

This Summary is provided to introduce a selection of concepts in a simplified form that are further described below in the Detailed Description. This Summary is not intended to identify key features or essential features of the claimed subject matter, nor is it intended to be used as an aid in determining the scope of the claimed subject matter.

Additional features and advantages will be set forth in the description which follows, and in part will be obvious from the description, or may be learned by the practice of the teachings herein. Features and advantages of the disclosure may be realized and obtained by means of the instruments and combinations particularly pointed out in the appended claims. Features of the present disclosure will become more fully apparent from the following description and appended claims or may be learned by the practice of the disclosure as set forth hereinafter.

### BRIEF DESCRIPTION OF THE DRAWINGS

In order to describe the manner in which the above-recited and other features of the disclosure can be obtained, a more particular description will be rendered by reference to specific embodiments thereof which are illustrated in the appended drawings. For better understanding, the like elements have been designated by like reference numbers throughout the various accompanying figures. While some of the drawings may be schematic or exaggerated representations of concepts, at least some of the drawings may be drawn to scale. Understanding that the drawings depict some example embodiments, the embodiments will be described and explained with additional specificity and detail through the use of the accompanying drawings in which:

FIG. 1 is a system diagram of a psychotherapy system including an HMD and a provider computing device, according to at least one embodiment of the present disclosure;

FIG. 2 is a rear perspective view of a head-mounted device for psychotherapy, according to at least one embodiment of the present disclosure;

FIG. 3 is a representation of a VR coordinator, according to at least one embodiment of the present disclosure;

FIG. 4-1 and FIG. 4-2 are representations of a graphical user interface (GUI) for a provider, according to at least one embodiment of the present disclosure;

FIG. 5 is a representation of a flowchart of a method for remote psychotherapy, according to at least one embodiment of the present disclosure; and

FIG. 6 is a representation of a schematic computing system, according to at least one embodiment of the present disclosure.

### DETAILED DESCRIPTION

The present disclosure relates generally to systems and methods for real-time biometric feedback during psychotherapy. More particularly, the present disclosure relates to real-time collection of electroencephalogram (EEG) information from a patient during a psychotherapy session. In at least one embodiment, the EEG information is collected via a head-mounted device (HMD) that also houses a near-eye display (NED) to provide visual information and/or stimulation to the patient during the psychotherapy session.

In some embodiments, a psychotherapy system according to the present disclosure allows a provider (such as a clinician, social worker, therapist, psychologist, psychiatrist, or another provider) to communicate with patients from a remote location via the internet or another data network. In some embodiments, the virtual environment (and elements therein) and avatars presented to the user with the HMD meet key descriptions or criteria for restorative environments and therapist characteristics, respectively. A therapy session is able to be conducted using a system described herein, and in some embodiments, the therapy session is conducted entirely using a system described herein. Major limitations relate to the difficulty of fostering a partnership between therapist and patient built on trust (i.e., the Therapeutic Alliance) when sessions are being conducted over a distance and the inability to control the environment in which the patient is undergoing sessions. The devices, systems, and methods described herein address one or more of these limitations.

A psychotherapy and EEG system according to the present disclosure includes an HMD with one or more biometric feedback sensor therein. In some embodiments, the HMD provides visual information and/or audio information to a patient, provides auditory information to the patient, and measures EEG information in real-time to measure the patient's response to the visual and auditory information. In some embodiments, the HMD is an integrated device with a NED, speakers, a microphone, at least one processor, and system memory containing instructions that, when executed by the processor, cause the HMD to perform any of the methods described herein. In some embodiments, the HMD is a headset that the user will place their smartphone into. The smartphone may include a display that functions as a NED when placed in the headset, and the smartphone may include speakers and a microphone to allow verbal conversations and talk therapy with the provider. The smartphone includes at least one processor, and system memory containing instructions that, when executed by the processor, cause the HMD to perform any of the methods described herein.

The system allows a provider to engage in real-time talk therapy with the patient through a virtual representation of the provider in the virtual environment and real-time conversations with a remote provider via the speakers and microphone. The HMD provides to the patient a first-person virtual therapy environment in which the patient can explore and then choose one of several environments designed based on the literature of healing environments, for example, a restorative natural mountain forest; a comfortable log cabin with a glowing hearth; or a professional therapy office. In some embodiments, the patient is able to select a provider avatar for the provider. In some embodiments, the provider is able to select a provider avatar for the provider. In some embodiments, the provider avatar is based on Jungian archetypes and the literature of effective therapist characteristics. In some embodiments, the patient avatar is not visible to the patient in the provided first-person perspective of the virtual environment. In some embodiments, the patient avatar is visible to the patient in the virtual environment. For example, the patient may view a portion of the patient avatar in the first-person perspective (such as hands, feet, or a reflection). In some examples, the HMD may provide a third-person perspective on the virtual environment in which the patient avatar is visible in the patient's field of view.

The provider avatar may move or have body or face animations to reflect actions of the provider. The provider avatar may, therefore, function as an interactive element in the virtual environment to the patient, with which the patient can direct conversations or other communications.

In some embodiments, the HMD includes one or more biometric feedback sensor to provide real-time biometric information to the provider and correlate that real-time biometric information with the visual and auditory information provided to the user. For example, the HMD may include EEG sensors positioned on an inner surface of the HMD to contact the patient's skin. In some embodiments, the EEG sensors are located around a periphery of the NED of the HMD to contact the patient's forehead, temples, cheeks, or facial skin. In some embodiments, the EEG sensors are positioned on a lateral headband or a crown headband to contact one or more locations on the patient's scalp. A separate ear clip will provide the ground for the EEG.

The EEG sensors of the HMD may provide patients and therapists with real time biofeedback during sessions. An EEG sensor will be incorporated into the VR headset and rest on the patient's forehead. The EEG readout provides information during therapy about the patient's emotional arousal, which is critical in the absence of visible nonverbal communication. In a local, in-person conversation, physical reactions such as facial expressions, microexpressions, posture changes, etc. are used to interpret patient reactions to visual or auditory information. For example, during a talk therapy session, visible reactions to the conversation provide important feedback to the provider. In a remote session, such visible nonverbal communication is lost, and a system according to the present disclosure provides the provider an alternative source of nonverbal communication. In some embodiments, the raw brain waves will also be available for observation in real time.

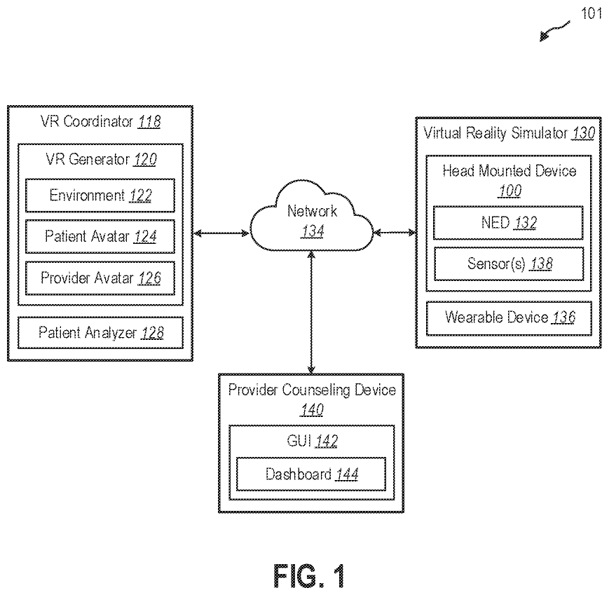

FIG. 1 is a representation of a remote psychotherapy system 101, according to at least one embodiment of the present disclosure. A remote server may include a virtual reality coordinator 118 may host a virtual reality session for a patient and a provider. For example, the virtual reality coordinator 118 may be located on a remote server, such as a cloud server or a parent server hosted by the virtual reality company. The virtual reality coordinator 118 may generate the virtual reality session using a VR generator 120. The VR generator 120 may generate the virtual reality session including an environment 122, a patient avatar 124, and a provider avatar 126. For example, the VR generator 120 may generate an environment 122. As discussed herein, the environment 122 may be developed to promote a therapeutic or restorative environment for the patient. For example, the environment 122 may be generated based on the principles of restorative environments. Examples of restorative environments may include, but are not limited to, a natural world (e.g., a forest, the mountains, the beach, the desert), a building (e.g., a log cabin, a cozy house), or a therapist's office (e.g., Freud's office, other comforting environment). Other restorative environments may also be utilized.

A user may provide input into the environment 122. For example, the virtual reality coordinator 118 may provide the user with an option to select the environment 122. In some embodiments, the user may select the general environment (e.g., the natural world, the building, the office). In some embodiments, the user may select specific details of the environment 122. For example, the user may select the type of trees, the decor of the house, or the layout of the therapist's office. Different patients may be comfortable in different environments. Because the therapy is being performed remotely, the user may control the environment 122 based on what he or she believes will be the most comfortable and therapeutic.

The VR generator 120 may prepare a patient avatar 124. The patient avatar 124 may be a virtual representation of the patient. The patient may modify aspects of the patient avatar 124 to be representative of an image the patient wishes to project. For example, the patient may customize the patient avatar 124 to be representative of the patient's real-body physical appearance. In some examples, the patient may customize the patient avatar 124 to be representative of the patient's idealized body or other imagined details.

The VR generator 120 may generate the virtual reality session with the patient avatar 124 located in the environment 122. This may allow the patient to interact with the environment 122. For example, through the patient avatar 124, the patient may walk through the environment 122 and/or interact with elements of the environment 122. This may allow the patient to experience the therapeutic benefits of the environment 122 without physically travelling to or being located at the environment 122.

The VR generator 120 may prepare a provider avatar 126. In some embodiments, the provider avatar 126 may be a virtual representation of the therapist. In some embodiments, the provider avatar 126 may be a fictionalized image. For example, the provider avatar 126 may be based on one or more Jungian archetypes. In some embodiments, the provider avatar 126 may be a virtual representation of a therapist that may match one or more characteristics that a patient may value and/or look for in a therapist. Examples of an archetypal provider avatar 126 may include the wise sage (e.g., a wise wizard, wise woman) and/or the healer (e.g., the healer woman).

In some embodiments, the provider avatar 126 may be provided based on key therapist characteristics. For example, the provider avatar 126 may be provided based on therapist characteristics that patients look for and trust. Key therapist characteristics may include expertness and wisdom, compassion and empathy, similarity and liking, and genuineness and trustworthiness.

In some embodiments, the patient may select one or more elements of the provider avatar 126. For example, the patient may feel a connection or trust with one or more of the archetypal provider avatars 126. When setting up the virtual reality session, the VR generator 120 may query the patient regarding a desired representation of the provider avatar 126. The patient may select one or more features, including a general archetype, skin color, hair type, hair color, and so forth. Using the patient-selected or patient-influenced provider avatar 126, the provider may facilitate a therapeutic conversation with the patient, including generating and/or improving on a relationship of trust.

The patient may interact with the virtual reality session using a virtual reality simulator 130. The virtual reality simulator 130 may be any virtual reality simulator in which a user may engage with a virtual reality environment, such as the environment 122 generated by the VR generator 120. In some embodiments, the virtual reality simulator 130 may allow the patient to generate, interact with, and manipulate an avatar in the virtual reality environment. For example, the virtual reality simulator 130 may allow the patient to build and manipulate a patient avatar 124 in the environment 122 hosted by the VR generator 120. In some embodiments, the virtual reality simulator 130 may include hardware and software the patient may use to interact with the virtual environment.

In accordance with at least one embodiment of the present disclosure, the virtual reality simulator 130 may include a head mounted device (HMD) 100. The HMD 100 may be any head mounted device that is configured to engage a patient or other user in a virtual reality environment. For example, the HMD 100 may include a gaming virtual reality headset, such as those headsets used for playing immersive virtual reality games. In some examples, the HMD 100 may include an industrial virtual reality headset, such as those headsets used to facilitate remote work, including remote medical care, remote equipment training and/or operation, remote education, and so forth.

The HMD 100 may include a near eye display (NED) 132. When the HMD 100 is mounted on the user's head, the NED 132 may be located in front of the user's eyes. In some embodiments, the NED 132 may be located close to the user's eyes. When generating the virtual reality session, the virtual reality simulator 130 may be connected to the virtual reality coordinator 118 via a network 134. The network 134 may be any network, such as the Internet, a local area network (LAN), any other network, and combinations thereof. The virtual reality coordinator 118 may transmit environmental information about the environment 122 and/or positional and other information about the provider avatar 126. The NED 132 may present a display of the environment 122. For example, the NED 132 may present a display of the environment 122 as viewed from the virtual position of the patient avatar 124.

In some embodiments, the virtual reality simulator 130 may include one or more wearable devices 136 for other body parts of the user. For example, the one or more wearable devices 136 may include one or more gloves, vests, pants, armbands, other wearable devices, and combinations thereof. The one or more wearable devices 136 may include feedback elements, such as haptics, which may allow the patient to receive feedback from the environment 122, including a sense of touching the various elements of the environment 122. In some embodiments, the one or more wearable devices 136 may include one or more input elements. The input elements may allow the user to control elements of the patient avatar 124, such as the movement of the patient avatar 124. The virtual reality simulator 130 may further include microphones and speakers which may further allow the patient to interact with the virtual reality session.

The HMD 100 may further include one or more sensors 138. The one or more sensors 138 may collect biofeedback information about the state of the user. The virtual reality simulator 130 may transmit the biofeedback information to the virtual reality coordinator 118 over the network 134. The virtual reality coordinator 118 may receive the biofeedback information at a patient analyzer 128. The patient analyzer 128 may analyze the biofeedback information to determine state information about the patient. For example, the patient analyzer 128 may review the biofeedback information to determine an emotional state or a physiological state of the patient. As discussed herein, the provider may utilize the determined state information to supplement or replace a physical, in-person analysis of the patient. For example, a provider may typically determine emotional state information of a patient using nonverbal cues, such as body posture. Because virtual therapy provides limited opportunities for nonverbal communication, the determined state information of the patient may help to supplement or replace such nonverbal cues. For example, the patient analyzer 128 may determine the emotional state of the patient using the biofeedback information. The provider may use this information in a therapy session to improve the level of care. In accordance with at least one embodiment of the present disclosure, the state information may include any state of the user, including emotional state, physiological state, medical state, any other state, and combinations thereof.

In accordance with at least one embodiment of the present disclosure, the sensor 138 may be any type of sensor. For example, the sensor 138 may be an electroencephalogram (EEG). The EEG may detect the electric activity of the patient's brain. Based on the detected electrical activity, the patient analyzer 128 may detect state information about the patient. For example, the patient analyzer 128 may use the detected brain electrical activity from the EEG to determine the emotional state of the patient. For example, the patient analyzer 128 may apply one or more filters and detect spikes at certain frequencies of the electrical activities (e.g., alpha, beta, gamma, delta, theta brain waves). These spikes may be associated with emotions. In this manner, using the EEG electrical activity, the provider may assess the emotional state of the patient. In some embodiments, the provider may assess the emotional state of the patient without visual and/or non-verbal cues from the patient.

In some embodiments, the sensor 138 may include any other type of biofeedback sensor, such as a pulse detector, a blood oxygen detector, a pulsimeter, a blood pressure monitor, a blood sugar detector, an electrocardiogram (ECG), pupil tracker, any other type of biofeedback sensor, and combinations thereof. In some embodiments, the virtual reality simulator 130 may include any number of sensor 138. In some embodiments, the virtual reality simulator 130 may include any combination of different types of sensors 138. In some embodiments, the sensor 138 may be incorporated into a portion of the HMD 100. For example, the sensor 138 may be incorporated into a headband of the HMD 100. In some embodiments, the sensor 138 may be incorporated into any other portion of the virtual reality simulator 130. For example, the sensor 138 may be incorporated into the one or more wearable devices 136. For example, a pulse oximeter may be incorporated into a wearable glove that includes haptics.

In some embodiments, a provider dashboard is stored on a remote server (e.g., the virtual reality coordinator 118) that allows recording and access of the virtual reality sessions, visual information from the patient's perspective in the virtual environment, auditory information from the patient's perspective in the virtual environment including audio recordings of the talk therapy session, EEG information with time correlations to the visual and/or auditory information, session notes, or other information about the patient history. The virtual reality simulator 130 may communicate with the remote server and the dashboard stored thereon via the network 134.

In accordance with at least one embodiment of the present disclosure, the provider may interact with the virtual reality session via a provider counseling device 140. In some embodiments, the provider counseling device 140 may be any type of counseling device. For example, the provider counseling device 140 may be a virtual reality device, such as a virtual reality headset and/or wearable devices uses in association with a virtual reality session. Using the provider counseling device 140, the provider may manipulate the provider avatar 126 and/or interact with the patient avatar 124.

In some embodiments, the provider counseling device 140 may include a graphical user interface (GUI) 142. The GUI 142 may include a provider dashboard 144. The provider dashboard 144 may be populated with patient information. For example, the provider dashboard 144 may include patient notes taken from previous therapy sessions. In some embodiments, the provider counseling device 140 may be in communication, via the network 134, with the virtual reality coordinator 118. The virtual reality coordinator 118 may provide patient information to the provider counseling device 140. For example, the virtual reality coordinator 118 may provide state information of the patient determined by the patient analyzer 128. The state information may be displayed on the provider dashboard 144. In this manner, as the provider is engaged in a virtual therapy session with the patient, the provider may review the state information of the patient. This may help the provider to determine the state of the patient without being in the same room or physically viewing the patient.

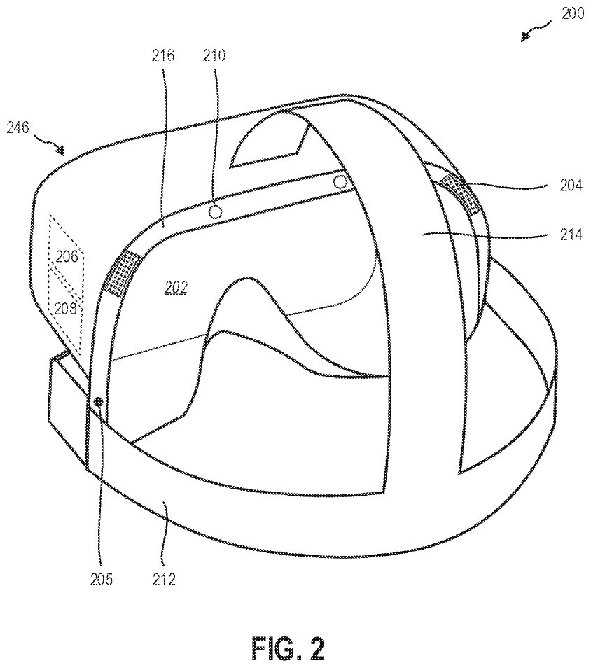

FIG. 2 is a rear perspective view of an HMD 200 according to at least some embodiments of the present disclosure. The HMD 200 has a NED 202, speakers 204, and a microphone 205 in communication with a processor 206. The NED 202 may be any type of display. The processor 206 accesses instructions on system memory 208 to perform any of the methods described herein. The HMD 200 also includes one or more EEG sensors 210 or other biofeedback sensors positioned on an inner surface of the HMD 200 to contact the patient's skin. For example, the EEG sensors 210 or other biofeedback sensors may be positioned on a lateral headband 212, a crown headband 214, a NED frame 216, or other locations.

The HMD 200 may include a housing 246. In accordance with at least one embodiment of the present disclosure, the housing 246 may be configured to allow a smart phone, tablet, or other computing device to be secured to the housing 246. The computing device may be secured to the housing 246 such that the display of the computing device functions as the NED 202. In some embodiments, the speakers 204 may be the speakers of the computing device, and/or the speakers 204 may be otherwise connected to the computing device. The computing device may further include the processor 206 and/or system memory 208. For example, the virtual reality environment may be rendered by the computing device on the display of the computing device. The computing device may be in communication with the one or more EEG sensors 210 This may help to reduce the cost of the HMD 100 to the user, because the patient likely already has smartphone or other suitable computing device.

In some embodiments, both therapist and patients will have dashboards that incorporate all of the information necessary for successful therapy. For example, a provider may have access to patient's avatar and environment preferences as well as patient history files. Providers may be able to store notes and observations from each of their sessions in the dashboard. In addition, the dashboard will include intake information and results of psychological assessments taken online by the patient.

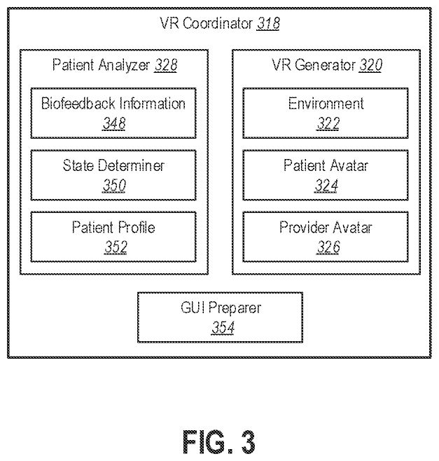

FIG. 3 is a representation of a VR coordinator 318, according to at least one embodiment of the present disclosure. The VR coordinator 318 includes a VR generator 320. The VR generator 320 may be configured to generate a virtual reality session between a patient and a provider. For example, the VR generator 320 may generate an environment 322. The VR generator 320 may develop a patient avatar 324 and a provider avatar 326. The patient avatar 324 and the provider avatar 326 may move around in and/or manipulate objects in the environment 322.

The VR coordinator 318 further includes a patient analyzer 328. The patient analyzer 328 may analyze information from the patient to determine state information about the patient. In some embodiments, the patient analyzer 328 may receive biofeedback information 348 from the user. For example, the patient analyzer 328 may receive the biofeedback information 348 from a sensor on an HMD, such as patient brain electrical activity measured from an EEG.

The patient analyzer 328 may include a state determiner 350 that reviews the biofeedback information 348 to determine state information about the user. For example, the state determiner 350 may review the brain electrical activity to determine the emotional or other state of the user. In some embodiments, the state determiner 350 may separate the electrical activity into frequency bands, such as alpha waves, beta waves, gamma, delta, theta waves, and so forth. The frequency bands themselves may be representative of or determinative of states. For example, Alpha waves (8 to 30 Hz) are often associated with relaxation, beta waves (12 to 30 Hz) are often associated with alertness, gamma waves (30+Hz) are often associated with creativity and flow, delta waves (0.1 to 3 Hz) are often associated dreamless sleep, and theta waves (4 to 7 Hz) are often associated with dreaming. The VR coordinator 318 may note deviations from these frequency bands. In some embodiments, the VR coordinator 318 may provide an alert to the provider regarding any deviations from these frequency bands. The alert may include a warning, such as to avoid a topic or to pursue a topic in further detail. The state determiner 350 may be trained to identify emotions and other state information by using machine learning techniques.

In some embodiments, the state determiner 350 may send to the provider the state information. For example, the state determiner 350 may send the determined emotional state to the provider. In some embodiments, the patient analyzer 328 may send the provider the raw biofeedback information 348, and the provider may determine state data from the biofeedback information 348. In some embodiments, the patient analyzer 328 may send partially processed biofeedback information. For example, the patient analyzer 328 may send the provider filtered biofeedback information, such as brain electrical activity filtered into alpha waves. This may allow the provider to determine emotional or other state information on his or her own.

In some embodiments, the patient analyzer 328 may maintain a patient profile 352 of the patient. Many therapeutic relationships occur over more than one therapy session. In a first (or other previous) therapy session, the patient analyzer 328 may receive the biofeedback information 348 and/or determined state information. The patient analyzer 328 may store the biofeedback information 348 and/or determined state information. This may allow a provider to review previous biofeedback information 348 and/or previous determined state information about the patient to facilitate patient healing.

In some embodiments, the state determiner 350 may review the stored biofeedback information 348 and/or determined state information when determining state information about the patient. Using the biofeedback information 348 and/or determined state information, the state determiner 350 may provide refined state information about the patient. For example, a provider may associate particular biofeedback information 348 with particular state information. The state determiner 350 may use this provider-based association to refine the state determination process.

In some embodiments, the biofeedback information 348 may be measured or correlated periodically. For example, the biofeedback information 348 may be measured or collected on a schedule, such as with a frequency of 0.1 Hz, 1 Hz, 5 Hz, 10 Hz, 15 Hz, 20 Hz, 30 Hz, 40 Hz, 50 Hz, 100 Hz, or any value therebetween. Periodically measuring the biofeedback information 348 may allow the provider to correlate the biofeedback information 348 (and/or state information determined using the biofeedback information 348) with communications between the patient and the provider, such as communications between the patient avatar and the provider avatar in the virtual environment. In some embodiments, periodically measuring the biofeedback information 348 may allow the provider to correlate the biofeedback information 348 (and/or state information determined using the biofeedback information 348) with how the patient interacts with the virtual environment. For example, the biofeedback information 348 may be correlated with interactions between the patient avatar and elements of the virtual environment (e.g., virtual trees, a virtual fireplace, virtual art).

In accordance with at least one embodiment of the present disclosure, the state determiner 350 may correlate the biofeedback information 348 from the sensor with communications between the patient and the provider (e.g., communications between the patient avatar and the provider avatar) and/or interactions of the patient with the virtual environment (e.g., interactions between the patient avatar and elements of the virtual environment). For example, the state determiner 350 may correlate specific patterns in the biofeedback information 348 (and/or associated state information) with keywords used by one or both of the patient or the provider. In some embodiments, the state determiner 350 may correlate patterns in biofeedback information 348 (and/or associated state information) with concepts discussed during a therapy session. For example, the state determiner 350 may correlate patterns in the biofeedback information 348 (and/or associated state information) with concepts discussed by one or both of the patient or the provider.

In some embodiments, the biofeedback information 348 may be measured or collected in correlation with a communication between the patient and the avatar (e.g., communications between the patient avatar and the provider avatar) and/or interactions of the patient with the virtual environment (e.g., interactions between the patient avatar and elements of the virtual environment). For example, the patient analyzer 328 may cause the biofeedback sensor on the patient's virtual reality simulator (e.g., the virtual reality simulator 130 of FIG. 1) to measure the biofeedback information 348 (and/or determine associated state information) upon a determination that there is a therapeutically significant communication occurring, such as a communication by the patient and/or the provider. In some embodiments, the patient analyzer 328 may cause the biofeedback sensor to measure the biofeedback information 348 (and/or determine associated state information) upon a determination that there is a therapeutically significant event occurring, such as an interaction between the patient avatar and the virtual environment. In some embodiments, the biofeedback information 348 may be measured in response to a signal from the provider. For example, the provider may desire to know the biofeedback information 348 of the patient in response to a communication by one or both of the patient or the provider.

In some embodiments, a provider may utilize the historical state information stored in the patient profile 352 during a therapy session to facilitate therapy. For example, while interacting with the patient via the patient avatar 324 and/or observing the patient avatar 324 interact with the environment 322, the provider may correlate state information with the interactions of the patient avatar 324. For example, the provider may correlate emotional state information with keywords and concepts used by the patient. This may help the provider to associate concepts with emotions or other states, which may improve the therapy process. In some embodiments, the provider may review specific biofeedback information 348 and/or state information based on correlations of the actions of the patient, such as interactions of the patient avatar 324 and the environment 322.

The VR coordinator 318 includes a graphical user interface (GUI) preparer 354. The GUI preparer 354 may collect information from the patient analyzer 328 to send to the provider. For example, the GUI preparer 354 may prepare a page template of a patient dashboard for the patient. The GUI preparer 354 may provide the prepared page template to the provider virtual reality device. The GUI preparer 354 may populate patient information on the GUI of the provider virtual reality device. For example, the page template may be populated with the biofeedback information 348 and/or the determined state data of the patient. In some embodiments, the page template may be populated with the biofeedback information 348 and/or the determined state data in real time. This may allow the provider to review the biofeedback information 348 and/or the determined state data during the virtual therapy session. This may allow the provider to determine the state of the patient to provide therapy based on the biofeedback information 348 and/or the determined state data.

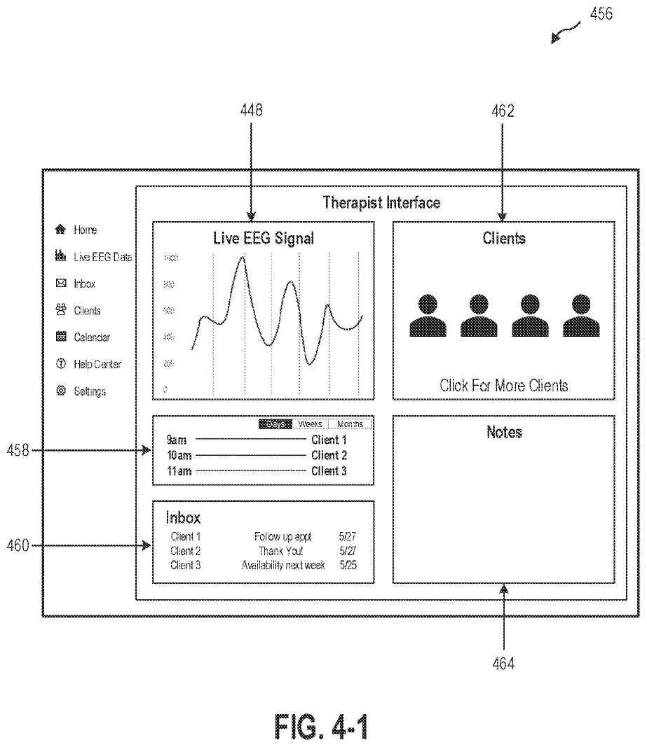

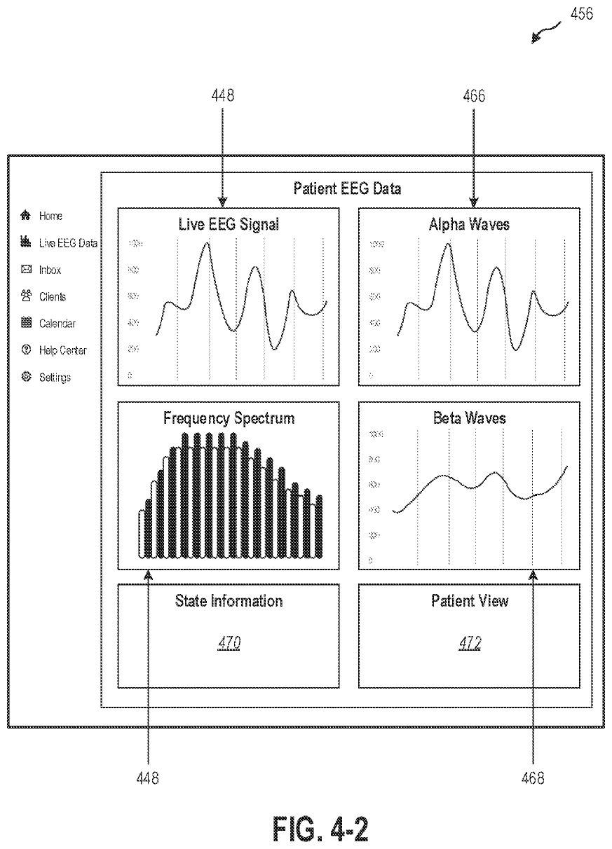

FIG. 4-1 is a representation of GUI having a presentation of a provider dashboard 456 with may be displayed on a provider virtual reality device, according to at least one embodiment of the present disclosure. The provider dashboard 456 may include multiple interactive icons and/or windows that display patient information. In the view shown in FIG. 4-1, the provider dashboard 456 includes an interactive icon of biofeedback information 448. The icon for the biofeedback information 448 may display measured biofeedback information 448. In some embodiments, the displayed biofeedback information 448 may be historical biofeedback information 448 for a particular patient. The provider may be reviewing the historical biofeedback information 448 prior to or after a virtual therapy session with the patient. In some embodiments, the displayed biofeedback information 448 may be live biofeedback information 448, such as the live signal from an EEG sensor on the patient's virtual reality device.

The provider dashboard 456 may include other information for the provider, such as a schedule or calendar 458. The calendar 458 may include the provider's schedule. The provider may select an interactive icon of a patient for a scheduled meeting (such as through touching a touchscreen or selecting with an input device such as a mouse). This may pull up patient information supplied by the VR coordinator (e.g., the VR coordinator 318 of FIG. 3). For example, when the provider selects an interactive icon for an appointment on the calendar 458, the biofeedback information 448 window may be populated with the biofeedback information 448 for the selected patient. The provider may select live or historical biofeedback information 448 to display in the biofeedback information 448 display.

The provider dashboard 456 may further include an inbox 460, a client list 462, and/or a section for notes 464. When the provider interacts with an interactive icon representing an email by a patient on the inbox 460, information regarding the patient may be populated in the biofeedback information 448 window. In some embodiments, the provider may select a picture of a client on the client list 462 to populate information on the biofeedback information 448 window. The provider may take notes in the notes 464. The notes may be patient-specific and may be associated with the same patient whose information is populated in the biofeedback information 448 window.

In FIG. 4-2, the provider dashboard 456 has been populated with detailed patient biofeedback information. The updated provider dashboard 456 may include the biofeedback information 448 presented in the provider dashboard 456 shown in FIG. 4-1. In some embodiments, the provider dashboard 456 may include analysis of the biofeedback information 448. For example, for EEG data, the provider dashboard 456 may include one or more filters of frequency ranges of the biofeedback information 448, such as alpha waves 466, beta waves 468, any other frequency range, and combinations thereof. In some embodiments, each filtered range may be populated in its own window. In some embodiments, the filtered ranges may be overlaid on a single window. In some embodiments, the biofeedback information 448 may be combined into a frequency spectrum plot 448.

Providing an analysis of the biofeedback information 448 may allow the provider to determine state information about the patient without being physically present in the same room as the user. For example, the provider may interpret the alpha waves 466 and/or the beta waves 468 to determine the emotional state of the patient without reviewing or analyzing non-verbal communication cues from the patient.

In some embodiments, the VR coordinator may provide state information 470 and populate it in the provider dashboard 456. The state information 470 may include any state of the patient, such as the emotional state, the physiological state, the chemical state, or other state information of the user. As discussed herein, the state information 470 may be based on the biofeedback information 448. The type of state information 470 provided may be based on the type of biofeedback information 448 measured. For example, emotional state information 470 may be determined based on EEG biofeedback information 448. Physiological state information 470 may be based on pulse and/or blood oxygen biofeedback information 448. Providing state information 470 may help the provider to determine the state of the patient without being physically present in the same room.

In some embodiments, the VR coordinator may provide the state information 470 in real-time. This may help to reduce the amount of time and/or concentration that the provider spends on determining the state information, thereby allowing the provider to focus on the patient. In some embodiments, the VR coordinator may provide correlations between the state information 470 and actions by the patient and/or the patient avatar. For example, the state information 470 may be correlated with keywords and/or concepts discussed by the patient (e.g., by the patient avatar), discussed by the provider (e.g., by the provider avatar), and/or discussed between the patient (e.g., the patient avatar) and the provider (e.g., the provider avatar).

In some embodiments, the VR coordinator may provide alerts to the provider in the state information 470 window. For example, the VR coordinator may analyze the state information of the patient in real time. The VR coordinator may provide alerts based on particular detected states. For example, the VR coordinator may provide an alert of a particular state, such as “High anxiety levels detected.” Providing state information in an alert may allow the VR coordinator to emphasize certain states for the provider. In some embodiments, the VR coordinator may provide suggestions to the therapist based on the detected state. For example, the VR coordinator may provide an alert that states “High anxiety levels detected: further probing on this topic could be beneficial.” As discussed herein, the VR coordinator may provide alerts based on historical state information. For example, the VR coordinator may provide an alert that states “High anxiety levels previously detected regarding this topic.” Furthermore, the VR coordinator may provide a suggestion, such as “High anxiety levels previously detected regarding this topic: further questioning along this topic may be problematic.”

In some embodiments, the provider dashboard 456 may further include previous state information. For example, the provider dashboard 456 may provide previous state information from a previous virtual therapy session. The previous state information may help the provider to determine current state information 470 of the patient. For example, the provider may associate the previous state information with actions by the patient, such as keywords or concepts discussed and/or interaction with objects in the virtual environment. In some embodiments, the previous state information may allow the provider to determine progress in therapy, such as by comparing an intensity of an emotional response to particular keywords, topics, or virtual interactions. In some embodiments, the previous state information may include previous biofeedback information.

In accordance with at least one embodiment of the present disclosure, the provider dashboard 456 may include a perspective of a patient view 472 from a patient. For example, the patient view 472 may include the view seen by the patient as the patient is manipulating the patient avatar. The provider may correlate the biofeedback information 448 and/or the state information 470 with the patient view 472. This may further help the provider to determine the state of the patient without being physically present with the patient.

In some embodiments, the VR coordinator may send the provider a report after the virtual therapy session. The report may include an analysis of the biofeedback information 448 and/or the determined state information 470. The report may further include elements of note, such as particularly strong determined states, transitions between states, or other therapeutic elements. In some embodiments, the report may include one or more correlations between the biofeedback information 448 and/or determined state information 470 and discussed keywords, concepts, and/or interactions between the patient avatar and the virtual environment. In some embodiments, the report may include correlations from the most recent virtual therapy session and previous virtual therapy sessions. Providing the provider with a report of the biofeedback information 448 and/or state information 470 may help the provider to review the virtual therapy sessions, identify state information 470 that the provider may not have noticed and/or not had time to address, and determine progress in state information 470 across sessions.

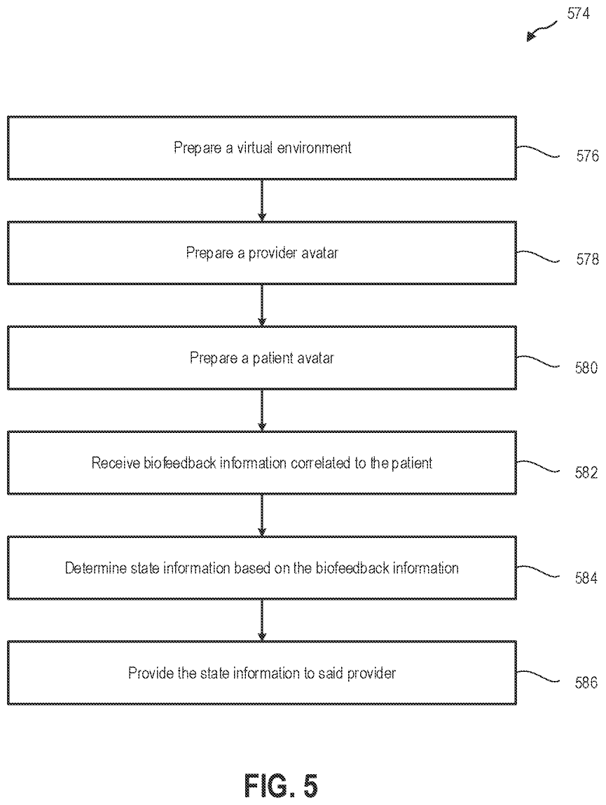

FIG. 5 is a flowchart of a method 574 for remote therapy, according to at least one embodiment of the present disclosure. A VR coordinator may prepare a virtual environment for a remote therapy session at 576. In some embodiments, the VR coordinator may prepare the virtual environment based on the receipt of a selection from the patient. The VR coordinator may prepare a provider avatar for a provider at 578. As discussed herein, the patient may have input regarding the provider avatar. In some embodiments, the VR coordinator may prepare the provider avatar based at least partially on a Jungian archetype. The VR coordinator may prepare a patient avatar for a patient at 580.

In some embodiments, the VR coordinator may receive biofeedback information from the patient that is correlated to the patient at 582. For example, the biofeedback information may be correlated to a communication to the patient from the provider avatar. In some examples, the biofeedback information may be correlated to the virtual environment. In some examples, the biofeedback information may be correlated to an interaction of the patient with the virtual environment.

In some embodiments, receiving the biofeedback information may include receiving electroencephalogram (EEG) sensor measurements. In some embodiments, the EEG sensor measurements may be provided to the provider.

In some embodiments, the method 574 may include determining state information for the patient based on the biofeedback information at 584. In some embodiments, determining the state information may include at least partially filtering the EEG sensor measurements. The filtered EEG sensor measurements may then be compared to historical measurements. In some embodiments, determining the state information may include determining an emotion of the patient based on the biofeedback information.

In some embodiments, the VR coordinator may provide the state information to the provider at 586. For example, as discussed herein, the VR coordinator may provide a page template of a presentation of a GUI to the provider. The VR coordinator may populate patient information (such as the biofeedback information and/or the state information) in the page template. The VR coordinator may then provide the page template, including the populated GUI, to the provider device.

In some embodiments, the method 574 may further include retrieving a user profile of the user. The user profile may include previous state information and/or previous biofeedback information. The VR coordinator may determine the state information based on a comparison of the previous state information or the previous biofeedback information with the state information or the biofeedback information of the current session.

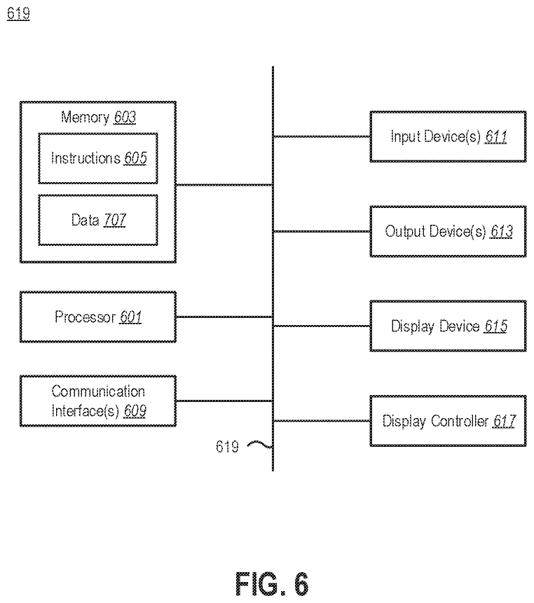

FIG. 6 illustrates certain components that may be included within a computer system 619. One or more computer systems 619 may be used to implement the various devices, components, and systems described herein.

The computer system 619 includes a processor 601. The processor 601 may be a general-purpose single or multi-chip microprocessor (e.g., an Advanced RISC (Reduced Instruction Set Computer) Machine (ARM)), a special purpose microprocessor (e.g., a digital signal processor (DSP)), a microcontroller, a programmable gate array, etc. The processor 601 may be referred to as a central processing unit (CPU). Although just a single processor 601 is shown in the computer system 619 of FIG. 6, in an alternative configuration, a combination of processors (e.g., an ARM and DSP) could be used.

The computer system 619 also includes memory 603 in electronic communication with the processor 601. The memory 603 may be any electronic component capable of storing electronic information. For example, the memory 603 may be embodied as random-access memory (RAM), read-only memory (ROM), magnetic disk storage media, optical storage media, flash memory devices in RAM, on-board memory included with the processor, erasable programmable read-only memory (EPROM), electrically erasable programmable read-only memory (EEPROM) memory, registers, and so forth, including combinations thereof.

Instructions 605 and data 607 may be stored in the memory 603. The instructions 605 may be executable by the processor 601 to implement some or all of the functionality disclosed herein. Executing the instructions 605 may involve the use of the data 607 that is stored in the memory 603. Any of the various examples of modules and components described herein may be implemented, partially or wholly, as instructions 605 stored in memory 603 and executed by the processor 601. Any of the various examples of data described herein may be among the data 607 that is stored in memory 603 and used during execution of the instructions 605 by the processor 601.

A computer system 619 may also include one or more communication interfaces 609 for communicating with other electronic devices. The communication interface(s) 609 may be based on wired communication technology, wireless communication technology, or both. Some examples of communication interfaces 609 include a Universal Serial Bus (USB), an Ethernet adapter, a wireless adapter that operates in accordance with an Institute of Electrical and Electronics Engineers (IEEE) 802.11 wireless communication protocol, a Bluetooth® wireless communication adapter, and an infrared (IR) communication port.

A computer system 619 may also include one or more input devices 611 and one or more output devices 613. Some examples of input devices 611 include a keyboard, mouse, microphone, remote control device, button, joystick, trackball, touchpad, and lightpen. Some examples of output devices 613 include a speaker and a printer. One specific type of output device that is typically included in a computer system 619 is a display device 615. Display devices 615 used with embodiments disclosed herein may utilize any suitable image projection technology, such as liquid crystal display (LCD), light-emitting diode (LED), gas plasma, electroluminescence, or the like. A display controller 617 may also be provided, for converting data 607 stored in the memory 603 into text, graphics, and/or moving images (as appropriate) shown on the display device 615.

The various components of the computer system 619 may be coupled together by one or more buses, which may include a power bus, a control signal bus, a status signal bus, a data bus, etc. For the sake of clarity, the various buses are illustrated in FIG. 6 as a bus system 619.

In accordance with at least one embodiment of the present disclosure, in contrast to eye movement desensitization and reprocessing (EMDR) or other bilateral stimulation techniques, the visual information provided by the HMD to the patient is represents a virtual environment in which the patient avatar is free to move. A provider avatar is also presented in the virtual environment to allow the patient a virtual representation of the provider with whom they are conversing. In some embodiments, each element of the virtual environments, avatars, and therapy modality is empirically based. For example, the elements of the virtual environments may be selected at least partially based on a large body of Common Factors experimental and meta-analytic research to support the selection. Common Factors are components of psychological healing strategies that are found across cultures and across psychotherapies.

In some embodiments, the virtual environment therapy setting in which the patient avatar and/or provider avatar are presented is chosen based at least partially on empirical research. For example, the virtual environment therapy setting may be a restorative environment, such as representations of nature that are found to reduce stress (including trees in close clusters, water movement, wide vistas, etc.). In some examples, the virtual environment therapy setting may be a healing environment, such as including components of indoor spaces associated with enhanced self-disclosure, perceptions of therapist trustworthiness, and relaxation (including lower lighting; rounded, soft furniture; 60-degree angled seating; natural textures; etc.).

The avatars, both the patient avatar and the provider avatar, may be chosen based on research on therapist characteristics linked to positive outcomes of therapy. For example, the avatars may exhibit design or animation characteristics that reflect properties such as expertness and wisdom, compassion and empathy, similarity and liking (to the patient), genuineness and trustworthiness, or other therapist characteristics. In at least one embodiment, an avatar includes design elements to reflect one or more Jungian archetype.

In some embodiments, the therapy rationale and method is based on the patient's worldview and diagnosis and has been found to have both relative and absolute efficacy in reducing distressing symptoms and promoting well-being in randomized, controlled trials. For example, the therapy rationale and method may include emotion-focused therapy, Cognitive Behavioral Therapy (CBT), Narrative therapy, Dialectical Behavioral Therapy, or other therapeutic modalities.

The articles “a,” “an,” and “the” are intended to mean that there are one or more of the elements in the preceding descriptions. The terms “comprising,” “including,” and “having” are intended to be inclusive and mean that there may be additional elements other than the listed elements. Additionally, it should be understood that references to “one embodiment” or “an embodiment” of the present disclosure are not intended to be interpreted as excluding the existence of additional embodiments that also incorporate the recited features. For example, any element described in relation to an embodiment herein may be combinable with any element of any other embodiment described herein. Numbers, percentages, ratios, or other values stated herein are intended to include that value, and also other values that are “about” or “approximately” the stated value, as would be appreciated by one of ordinary skill in the art encompassed by embodiments of the present disclosure. A stated value should therefore be interpreted broadly enough to encompass values that are at least close enough to the stated value to perform a desired function or achieve a desired result. The stated values include at least the variation to be expected in a suitable manufacturing or production process, and may include values that are within 5%, within 1%, within 0.1%, or within 0.01% of a stated value.

A person having ordinary skill in the art should realize in view of the present disclosure that equivalent constructions do not depart from the spirit and scope of the present disclosure, and that various changes, substitutions, and alterations may be made to embodiments disclosed herein without departing from the spirit and scope of the present disclosure. Equivalent constructions, including functional “means-plus-function” clauses are intended to cover the structures described herein as performing the recited function, including both structural equivalents that operate in the same manner, and equivalent structures that provide the same function. It is the express intention of the applicant not to invoke means-plus-function or other functional claiming for any claim except for those in which the words ‘means for’ appear together with an associated function. Each addition, deletion, and modification to the embodiments that falls within the meaning and scope of the claims is to be embraced by the claims.

It should be understood that any directions or reference frames in the preceding description are merely relative directions or movements. For example, any references to “front” and “back” or “top” and “bottom” or “left” and “right” are merely descriptive of the relative position or movement of the related elements.

The present disclosure may be embodied in other specific forms without departing from its spirit or characteristics. The described embodiments are to be considered as illustrative and not restrictive. The scope of the disclosure is, therefore, indicated by the appended claims rather than by the foregoing description. Changes that come within the meaning and range of equivalency of the claims are to be embraced within their scope.

What is claimed is:

1. A psychotherapy system comprising:
a head-mounted device (HMD) including:
a near-eye display (NED);
a speaker;
a microphone;
at least one biofeedback sensor;
a processor in communication with the NED, the speaker, the microphone, and the at least one biofeedback sensor; and
a system memory having instructions stored thereon that, when executed by the processor, cause the HMD to:
display a virtual environment including a provider avatar;
allow a patient to communicate with the provider avatar in real-time via the speaker and microphone using a patient avatar; and
measure at least one biofeedback information using the at least one biofeedback sensor correlated to a communication between the provider avatar and the patient avatar or the virtual environment.

2. The psychotherapy system of claim 1, wherein the at least one biofeedback sensors is an electroencephalogram (EEG).

3. The psychotherapy system of claim 1, further comprising a remote server in communication with the HMD, the instructions further including transmitting visual information, audio information, and biometric information from the HMD to the remote server for determination of state data correlated.

4. The psychotherapy system of claim 1, wherein the NED, the speaker, and the microphone are included as part of a computing device separate from the HMD.

5. The psychotherapy system of claim 4, wherein the HMD includes a housing, and wherein the computing device is configured to be secured to the housing.

6. A method for remote psychotherapy, comprising:
preparing a virtual environment for a remote therapy session;
preparing a provider avatar for a provider;
preparing a patient avatar for a patient;
receiving biofeedback information from said patient correlated to a communication to the patient from the provider avatar or the virtual environment;
determining state information about said patient based on the biofeedback information; and
providing the state information to said provider.

7. The method of claim 6, wherein the provider avatar is based at least partially on a Jungian archetype.

8. The method of claim 6, wherein preparing the virtual environment includes receiving a selection from said patient.

9. The method of claim 6, wherein said provider is located remotely from said patient.

10. The method of claim 6, wherein receiving the biofeedback information includes receiving electroencephalogram (EEG) sensor measurements.

11. The method of claim 10, further comprising providing the EEG sensor measurements to said provider.

12. The method of claim 10, wherein determining the state information includes at least partially filtering the EEG sensor measurements and comparing the filtered EEG sensor measurements to historical measurements.

13. The method of claim 6, wherein determining the state information includes determining an emotion of said patient based on the biofeedback information.

14. The method of claim 6, further comprising retrieving a user profile including at least one of previous state information or previous biofeedback information, and wherein determining the state information includes comparing the at least one of the previous state information or the previous biofeedback information with at least one of the state information or the biofeedback information.

15. The method of claim 14, further comprising:
providing a page template of a graphical user interface (GUI) of a provider device to said provider; and
populating at least one of the biofeedback information or the state information in the page template.

16. A remote psychotherapy system, comprising:
a memory and processor, the memory including instructions which, when accessed by the processor, cause the processor to:
prepare a virtual environment for a remote therapy session;
prepare a provider avatar for a provider;
prepare a patient avatar for a patient;
receive biofeedback information from said patient correlated to a communication to the patient from the provider avatar or the virtual environment;
determine state information about said patient based on the biofeedback information; and
provide the state information to said provider.

17. The system of claim 16, wherein the provider avatar is based at least partially on a Jungian archetype.

18. The system of claim 16, wherein said provider is located remotely from said patient.

19. The system of claim 16, wherein receiving the biofeedback information includes receiving electroencephalogram (EEG) sensor measurements.

20. The system of claim 19, wherein determining the state information includes at least partially filtering the EEG sensor measurements and comparing the filtered EEG sensor measurements to historical measurements.
